(19) United States
(12) Patent Application Publication
(10) Pub. No.: US 2005/0118273 A1
(43) Pub. Date: Jun. 2, 2005

(54) Microcapsules and oral composition containing the same

(76) Inventors: Yasushi Sasaki, Osaka (JP); Orikoshi Hideyuki, Osaka (JP); Hideo Hayashi, Osaka (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/494,515

(22) PCT Filed: Nov. 15, 2002
(86) PCT No.: PCT/JP02/11940

(30) Foreign Application Priority Data
Nov. 15, 2001 (JP) 2001-350836

(51) Int. Cl. A61K 9/68; A61K 9/16; A61K 9/50

(52) U.S. Cl. 424/490; 424/48; 424/93.4

(57) ABSTRACT

The present invention provides microcapsules with favorable speed, intensity and persistence of the release of foreign substances encapsulated inside microorganism cells. The present invention also provides a manufacturing method of a microcapsule and an oral composition containing the microcapsule. The microcapsules having at least one foreign substance enclosed in microorganism cells onto whose surfaces are adhered at least one member selected from the group consisting of saccharides, high-intensity sweeteners, proteins and polyhydric alcohols.

### TECHNICAL FIELD

The present invention relates to microcapsules of foreign substances encapsulated inside microorganism cells. More specifically, the invention relates to microcapsules of foreign substances encapsulated inside microorganism cells, wherein the speed, intensity and persistence of the release of foreign substances can be controlled. The present invention further relates to a manufacturing method for these microcapsules and to an oral composition containing the same.

### BACKGROUND ART

So-called microencapsulation methods known in the art involve enclosing a desired component inside particle containers (microcapsules) made of natural or synthetic macromolecules and having a diameter of from a few to several hundred μm in order to control the release speed and the release times thereof so as to increase its persistence. Well-known microencapsulation methods include coacervation methods using gelatin (e.g. U.S. Pat. Nos. 2,800,457, 2,800,458), in-situ methods involving the building of an outer (aqueous phase) membrane (e.g. Japanese Published Examined Application Nos. S36-9168, S47-23165, Japanese Published Unexamined Application Nos. S48-57892, S51-9079, S54-25277), and interfacial polymerization methods using a film-forming reaction between the inner and outer phases; in recent years microorganism microencapsulation methods wherein the cell membranes of microorganism such as yeast are used as capsule films have also become widely known.

Microcapsule manufacturing methods using microorganism cells include methods wherein flavorings or other desired foreign substances are encapsulated inside yeast cells without removing the contents of the cells (as disclosed for example in Japanese Published Unexamined Application Nos. S61-88871, S62-186937, S63-88033, S58-107189, and WO94/22572, WO96/36433, EP 0242135B1, etc.), as well as methods wherein the desired foreign substances are encapsulated in yeast cells after removing the endogenous intracellular components (as disclosed for example in Japanese Published Unexamined Application Nos. H4-4033, H4-63127, H4-117245, H5-15770, H8-243378, etc.). The latter method is advantageous in that the greater the amount of endogenous intracellular components that can be removed from the yeast cells, the greater the amount of foreign substances that can be encapsulated therein, thereby making it possible to increase the effect of these foreign substances.

In the food industry, for example, chewing gum is a kind of product that demands a rapid but sustained release of taste and aroma (flavor), which is difficult to achieve in practice, since although taste and flavor are brought forth intensely upon initial chewing, it tends to become rapidly weaker as chewing proceeds. Therefore, methods allowing the flavor to be sustained as long as possible have been actively studied. Japanese Published Unexamined Application No H5-192085 proposes a method for preparing gum using a flavoring agent obtained by enclosing a flavoring inside a microorganism cell thereby making it possible to obtain a chewing gum with a strong and sustained flavor, using a small amount of flavoring.

However, the release speed, intensity and persistence of flavorings or spices (sweeteners, etc.) as foreign substances (constituents) enclosed in microorganism cells are by no means yet sufficient and requires further improvement.

### DISCLOSURE OF THE INVENTION

The purpose of the present invention is to provide microcapsules of foreign substances encapsulated inside microorganism cells, and also to improve the release properties of these encapsulated foreign substances (constituents), increasing the speed, intensity and persistence of the release of constituents. A further purpose of the present invention is to provide an oral composition wherein the effect of such encapsulated foreign substances can be achieved more efficiently with smaller amounts of foreign substances, or an oral composition having an excellent persistence of the encapsulated foreign substances with smaller amounts of foreign substances, by using the aforementioned microcapsules.

As a result of a diligent study towards solving the above problems, the present inventors discovered that in so-called microorganism microcapsules of foreign substances encapsulated inside microorganism cells, by partially or completely covering these microorganism cells (capsules) with at least one member selected from the group consisting of saccharides, high-intensity sweeteners, proteins and polyhydric alcohols, it is possible to improve the speed, intensity and persistence of the release of these foreign substances while preventing their degradation. The inventors also noticed that these effects are notably improved by using microorganism cells whose endogenous intracellular component has been previously removed for encapsulating foreign substances. The present invention is achieved based on these findings.

Specifically, the present invention provides microcapsules including Items 1 to 9 below.

Item 1.

Microcapsules having foreign substances encapsulated inside microorganism cells onto whose surfaces are adhered at least one member selected from the group consisting of saccharides, high-intensity sweeteners, proteins and polyhydric alcohols.

Item 2.

The microcapsules according to Item 1, wherein the microorganism cells are that have been previously treated to remove (including “remove by elution”) the endogenous intracellular components.

Item 3.

The microcapsules according to Item 1 or 2, wherein the microorganism cells are yeast cells.

Item 4.

The microcapsules according to any one of Items 1 to 3, wherein the saccharides are at least one member selected from the group consisting of monosaccharides, disaccharides, trisaccharides, tetrasaccharides, oligosaccharides, polysaccharides and sugar alcohols.

Item 5.

The microcapsules according to any one of Items 1 to 3, wherein to the surfaces of the microorganism cells are adhered at least one member selected from the group consisting of lactose, maltitose, trehalose, palatinit, dextrin, glycerin, propylene glycol, polyglycerin, and polypropylene glycol.

Item 6.

The microcapsules according to any one of Items 1 to 5, wherein the foreign substance encapsulated in the microorganism cells is a hydrophobic substance.

Item 7.

The microcapsules according to any one of Items 1 to 6, wherein the foreign substance encapsulated in the microorganism cells is at least one member selected from the group consisting of flavorings, colorings, bitterants, acidulants, seasonings, spices, sweeteners, fats/lipids, fatty acids, amino acids, enzymes, vitamins, animal and plant extracts, and bioactive substances.

Item 8.

The microcapsules according to any one of Items 1 to 7, which is used as a constituent in foods, pharmaceuticals, quasi-pharmaceuticals, cosmetics or feeds.

Item 9.

The microcapsules according to any one of Items 1 to 8, wherein the surfaces of the microorganism cells encapsulating the foreign substance are covered with at least one member selected from the group consisting of saccharides, high-intensity sweeteners, proteins and polyhydric alcohols.

These microcapsules include the following embodiments:

(a) The microcapsules according to any one of Items 1 to 9 used as a flavoring wherein the foreign substance encapsulated in the microorganism cells is at least a flavoring.

(b) The microcapsules according to any one of Items 1 to 9 used as a sweetener wherein the foreign substance encapsulated in the microorganism cells is at least a sweetener.

(c) The microcapsules according to any one of Items 1 to 9 used as a colorant wherein the foreign substance encapsulated in the microorganism cells is at least a coloring.

(d) The microcapsules according to any one of Items 1 to 9 used as a seasoning wherein the foreign substance encapsulated in the microorganism cells is at least a seasoning.

(e) The microcapsules according to any one of Items 1 to 9 used as an acidulant wherein the foreign substance encapsulated in the microorganism cells is at least an acidulant.

(f) The microcapsules according to any one of Items 1 to 9 used as a bitterant wherein the foreign substance encapsulated in the microorganism cells is at least a bitterant.

(g) The microcapsules according to any one of Items 1 to 9 used as a spice wherein the foreign substance encapsulated in the microorganism cells is at least a spice.

The present invention also provides a method for manufacturing the aforementioned microcapsules including Items 10 to 11 below.

Item 10.

A manufacturing method of microcapsule comprising the steps of:
(1) encapsulating a foreign substance inside microorganism cells, and
(2) adhering onto the surface of the microorganism cells obtained in step (1) at least one member selected from the group consisting of saccharides, high-intensity sweeteners, proteins and polyhydric alcohols.

Item 11.

The manufacturing method of microcapsule according to Item 10 wherein step (1) is a step of encapsulating a foreign substance inside microorganism cells whose endogenous contents have been removed.

These microcapsule manufacturing methods include the following embodiments:

(a) The manufacturing method of microcapsule according to Item 10 or 11 using yeast cells as the microorganism cells.

(b) The manufacturing method of microcapsule according to Item 10 or 11, wherein the saccharide adhered to the surfaces of the microorganism cells is at least one member selected from the group consisting of monosaccharides, disaccharides, trisaccharides, tetrasaccharides, oligosaccharides, polysaccharides and sugar alcohols.

(c) The manufacturing method of microcapsule according to Item 10 or 11, wherein to the surfaces of the microorganism cells are adhered at least one member selected from the group consisting of lactose, maltitose, trehalose, palatinit, dextrin, glycerin, propylene glycol, polyglycerin, and polypropylene glycol.

(d) The manufacturing method of microcapsule according to Item 10 or 11, wherein the foreign substance encapsulated in the microorganism cells is a hydrophobic substance.

(e) The manufacturing method of microcapsule according to Item 10 or 11, wherein the foreign substance encapsulated in the microorganism cells is at least one member selected from the group consisting of flavorings, colorings, bitterants, acidulants, seasonings, spices, sweeteners, fats/lipids, fatty acids, amino acids, enzymes, vitamins, animal/plant extracts and bioactive substances.

(f) The manufacturing method of microcapsule according to Item 10 or 11, wherein step (2) is a step of covering the surface of the microorganism cell with at least one member selected from the group consisting of saccharides, high-intensity sweeteners, proteins and polyhydric alcohols.

The present invention further provides an oral composition containing microcapsules according to any one of Items 1 to 9. This oral composition may be an edible composition such as a food, oral pharmaceutical, oral quasi-pharmaceutical, etc., or an oral cavity composition such as a dentifrice, etc. The oral composition is preferably a food. Suitable oral compositions herein include, but are not limited to, gums such as chewing gum, soft candy such as gummy and nougat, and other items which are held for a prolonged time in the mouth such as masticatories, etc.

Item 12.

An oral composition containing microcapsules of any one of Items 1 to 9.

Item 13.

The oral composition according to Item 12, which contains microcapsules of any one of Items 1 to 9 as at least one member selected from the group consisting of flavorings, colorings, seasonings, acidulants, bitterants, spices and sweeteners.

Item 14.

The oral composition according to Item 12 or 13, which is held or chewed in the mouth for a prolonged time.

Item 15.

The oral composition according to any one of Items 12 to 14, wherein the oral composition is a chewing gum.

The present invention also provides a method for controlling the release of foreign substances encapsulated in the microcapsules, including Items 16 to 20 below:

Item 16

A method for controlling the speed, intensity or persistence of foreign substances released from microcapsules of the foreign substances encapsulated in microorganism cells, comprising the step of adhering onto the surface of these microcapsules at least one member selected from the group consisting of saccharides, high-intensity sweeteners, proteins and polyhydric alcohols.

Item 17

The method according to Item 16, which is a method for controlling the speed, intensity or persistence of the expression of flavor/taste from microcapsules of a flavor/taste component encapsulated in the microorganism cells.

Item 18

The method according to Item 17, wherein the microcapsules of the flavor/taste component encapsulated in the microorganism cells are prepared by encapsulating a flavor/taste component in microorganism cells whose endogenous intracellular components have been removed.

Item 19

The method according to Item 17 or 18, which is a method for increasing the speed, intensity or persistence of the expression of flavor or taste from the microcapsules.

Item 20.

The method according to any one of Items 17 to 19 comprising the step of covering the surfaces of the microorganism cells with at least one member selected from the group consisting of saccharides, high-intensity sweeteners, proteins and polyhydric alcohols.

These controlling methods include the following embodiments:

(a) The method according to any one of Items 16 to 20 using yeast cells as the microorganism cells.

(b) The method according to any one of Items 16 to 20, wherein the saccharides adhered to the surfaces of the microorganism cells is at least one member selected from the group consisting of monosaccharides, disaccharides, trisaccharides, tetrasaccharides, oligosaccharides, polysaccharides and sugar alcohols.

(c) The method according to any one of Items 16 to 20, wherein to the surfaces of the microorganism cells are adhered to by least one compound selected from the group consisting of lactose, maltitose, trehalose, palatinit, dextrin, glycerin, propylene glycol, polyglycerin, and polypropylene glycol.

(d) The method according to any one of Items 16 to 20, wherein the foreign substance encapsulated in the microorganism cells is a hydrophobic substance.

(e) The method according to any one of Items 16 to 20, wherein the foreign substance encapsulated in the microorganism cells is at least one member selected from the group consisting of flavorings, colorings, bitterants, acidulants, seasonings, spices, sweeteners, fats/lipids, fatty acids, amino acids, enzymes, vitamins, animal and plant extracts, and bioactive substances.

The present invention also provides a method for controlling the speed, intensity and persistence of the expression of a component in an oral composition, including Items 21 to 25 below:

Item 21.

A method for controlling the speed, intensity and persistence of the expression of a foreign substance in an oral composition, including oral compositions containing microcapsules of foreign substances encapsulated inside microorganism cells onto whose surfaces are adhered at least one member selected from the group consisting of saccharides, high-intensity sweeteners, proteins and polyhydric alcohols.

Item 22.

A method for controlling the speed, intensity and persistence of the expression of flavor or taste in an oral composition, including oral compositions containing microcapsules of flavor/taste components encapsulated inside microorganism cells onto whose surfaces are adhered at least one member selected from the group consisting of saccharides, high-intensity sweeteners, proteins and polyhydric alcohols.

Item 23.

The method according to Item 21 or 22, wherein the microorganism cells are that have been previously treated to remove the endogenous intracellular components.

Item 24.

The method according to any one of Items 21 to 23, which increases the speed, intensity or persistence of the expression of a foreign substance, in particular flavor or taste, in an oral composition.

Item 25.

The method according to any one of Items 21 to 24, which uses microorganism cells onto whose surfaces are covered with at least one member selected from the group consisting of saccharides, high-intensity sweeteners, proteins and polyhydric alcohols as microcapsules.

These methods include the following embodiments:

(a) The method according to any one of Items 21 to 25, which uses yeast cells as the microorganism cells.

(b) The method according to any one of Items 21 to 25, wherein the saccharides adhered to the surface of the microorganism cell are at least one member selected from the group consisting of monosaccharides, disaccharides, trisaccharides, tetrasaccharides, oligosaccharides, polysaccharides and sugar alcohols.

(c) The method according to any one of Items 21 to 25, wherein to the surface of the microorganism cell is adhered at least one member selected from the group consisting of lactose, maltitose, trehalose, palatinit, dextrin, glycerin, propylene glycol, polyglycerin, and polypropylene glycol.

(d) The method according to any one of Items 21 to 25, wherein the foreign substance encapsulated in the microorganism cell, in particular a flavor/taste component is a hydrophobic substance.

(e) The method according to Item 21, wherein the foreign substance encapsulated in the microorganism cells is at least one member selected from the group consisting of flavorings, colorings, bitterants, acidulants, seasonings, spices, sweeteners, fats/lipids, fatty acids, amino acids, enzymes, vitamins, animal and plant extracts, and bioactive substances.

(f) The method according to any one of Items 22 to 25, wherein the flavor/taste components encapsulated in the microorganism cells are at least one member selected from the group consisting of flavorings, colorings, bitterants, acidulants, seasonings, spices, sweeteners, fats/lipids, fatty acids, amino acids, enzymes, vitamins, or animal or plant extracts.

The present invention further includes the following Items:

Item 26.

Use of microcapsules having foreign substances encapsulated inside microorganism cells onto whose surfaces are adhered at least one member selected from the group consisting of saccharides, high-intensity sweeteners, proteins and polyhydric alcohols, as flavorings, sweeteners, colorings, seasonings, bitterants, spices or acidulants.

Item 27

Use of microcapsules having flavor/taste components encapsulated inside microorganism cells onto whose surfaces are adhered at least one member selected from the group consisting of saccharides, high-intensity sweeteners, proteins and polyhydric alcohols, for the preparation of flavorings, sweeteners, seasonings, bitterants, spices, or acidulants having an improved speed, intensity and persistence of the expression of flavor or taste.

Item 28

Use of microcapsules having foreign substances encapsulated inside microorganism cells onto whose surfaces are adhered at least one member selected from the group consisting of saccharides, high-intensity sweeteners, proteins and polyhydric alcohols, for the preparation of an oral composition.

Item 29

Use of microcapsules having flavor/taste components encapsulated inside microorganism cells onto whose surfaces are adhered at least one member selected from the group consisting of saccharides, high-intensity sweeteners, proteins and polyhydric alcohols, for the preparation of an oral composition having an improved speed, intensity and persistence of the expression of flavor or taste.

Item 30.

Use of microcapsules having flavor/taste components encapsulated inside microorganism cells onto whose surfaces are adhered at least one member selected from the group consisting of saccharides, high-intensity sweeteners, proteins and polyhydric alcohols, for increasing the speed, intensity and persistence of the expression of flavor or taste in an oral composition.

### BEST MODE FOR CARRYING OUT THE INVENTION

(1) Microcapsules

The microcapsules according to the present invention are microcapsules of foreign substances encapsulated inside microorganism cells onto whose surfaces are adhered at least one member selected from the group consisting of saccharides, high-intensity sweeteners, proteins and polyhydric alcohols.

Microorganism used in the present invention for encapsulating foreign substances include any kind of microorganism, for example algae, yeasts, fungi and bacteria, provided they have cell walls and their safety towards humans is proven. Preferred herein are yeasts. Yeasts that may be used include yeasts that can be properly ingested by the human body such as brewer's yeasts, baker's yeasts, torula yeasts, etc. Specific examples of yeasts include for example yeasts belonging to Saccharomyces such as Saccharomyces cerevisiae, Saccharomyces rouxii, Saccharomyces carlsbergensis, etc. and Candida yeasts such as Candida utilis, Candida tropicalis, Candida lipolytica, Candida flaveri, etc. Such microorganism cells may be used singly or in combination of 2 or more. These microorganism cells are not restricted in any particular way, but their particle size ranges preferably from 1 μm to 20 μm.

These microorganism cells may be used in any state for encapsulating the foreign substances, as viable or dead microorganism cells, in a wet or dry state, and with or without their endogenous intracellular component removed by elution. Microorganism cells with their endogenous intracellular components removed (including removed by elution) beforehand are preferably used. Endogenous intracellular components herein include amino acids, peptides, proteins (including enzymes), sugars, nucleic acids, fats/lipids, etc. Elution of endogenous intracellular components allows herein enclosing more foreign substances inside the microorganism cells while significantly suppressing undesirable smells and tastes elicited by the endogenous intracellular components and also preventing any possible decomposition or denaturation of the foreign substances brought about by the endogenous intracellular component.

The method for eluting the intracellular components (endogenous content) is not particularly restricted and may include methods known in the art or methods according to future developments. Conventional methods include for example physical treatment methods involving thermal treatment, pH treatment and cell wall crushing; chemical methods involving the addition of elution promoting agents; enzymatic methods using enzymes for dissolving out the intracellular component or enzymes for cell-wall lysis, and combinations of these methods (Japanese Published Unexamined Application No. H4-4033).

Thermal treatment herein can be carried out by heating a suspension of the target microorganism cells usually at 30 to 100° C., preferably at 30 to 60° C., while stirring for several minutes to several hours. Elution promoting agents may be simultaneously used in order to increase the efficiency of the elution of the intracellular component. Suitable elution promoting agents include for example polar organic solvents such as ethanol, propanol and other lower alcohols, ethyl acetate, acetone, and other polar organic solvents; inorganic bases, sugars, quaternary ammonium salts, various kinds of fungicides, bactericides, germicides, and bases such as sodium hydroxide, potassium hydroxide, etc. A specific example of such a method involves adding a solvent such as acetone or the like to an aqueous dispersion of the microorganism and then shaking at about 40° C. for about 24 hours.

In cell-wall crushing treatments, the breaking of the cell walls may be accomplished using a sonicator, a mill, etc. A specific example of such a method involves treating an aqueous dispersion of the microorganism for 10 minutes in a bead mill.

Enzymatic methods using enzymes for dissolving out the intracellular components may involve the use of autolytic enzymes from the microorganism itself (Babayan, T. L. and Bezrukov, M. G., 1 Acta Biotechnol. 0,5,129-136 (1985)), and enzymatic treatment methods with protease alone, or protease in combination with at least one selected from nuclease, β-glucanase, esterase and lipase. A concrete way of implementing such a method may involve for example incubating for 1 to 48 hours at 30 to 60° C. an aqueous dispersion of the microorganism possessing autolytic enzymes, or an aqueous dispersion of a microorganism to which the aforementioned enzymes have been added.

Enzymatic treatments for cell-wall lysis involve treating the microorganism cells using for example at least one type of enzyme such as glucanase (β-1,3-glucanase), mannanase, chitinase, etc. that decomposes the constituents of the microorganism's cell wall (polysaccharides such as glucan and mannan, complexes of these polysaccharides and proteins, chitin, etc.). A concrete way of implementing such an enzymatic treatment for cell-wall lysis may involve for example incubating an aqueous dispersion of the microorganism to which has been added the aforementioned enzymes for several minutes to about 10 hours at 30 to 60° C., ordinarily at a pH from 4 to 9.

The material thus obtained may be further centrifuged, discarded the supernatant, while the residue (cells) is subsequently washed, heated and/or pH adjusted as needed, in order to obtain microorganism cells (variants) with their intracellular component removed.

In those microorganism cells with their endogenous intracellular components eluted used in the present invention, the elution method and the extent of elution are not restricted to the aforementioned examples, but is meant to include a wide range of other methods wherein the intracellular constituents are also removed by elution. Microorganism cells with their endogenous cell components eluted include herein preferably microorganism cells wherein the proportion of absolute dry weight of eluted component (elution rate) relative to 100 wt % of absolute dry weight of untreated microorganism cells ranges from 10 to 80 wt %, and more preferably from 30 to 70 wt %.

Commercially available products may be conveniently used as microorganism cells with their endogenous intracellular components (endogenous components) removed. Such products include for example, but are not restricted to, commercially available yeast cell walls from Oriental Yeast, Co., Ltd.; Tanabe Seiyaku Co. Ltd.; Asahi Food & Healthcare Ltd.; Kirin Brewery Co. Ltd., etc. (all Japanese firms).

In order to encapsulate as much foreign substance as possible inside these microorganism cells (cell residues) having had their endogenous intracellular component removed, further treatments may be performed as needed in addition to those listed above, for example acid treatments (Japanese Published Unexamined Application No. H8-243378), alkali treatments (Japanese Published Examined Application No. H7-32871), alcohol treatments (Japanese Published Examined Application No. H8-29246), etc. Such aforementioned methods known in the art are not restricted, being limited only by the allowable physics and chemistry of the prepared microorganism cell walls acting as microcapsule films.

In an acid treatment, specifically, the cell residues obtained by enzymatic treatment or by other methods above are suspended for example in an acidic aqueous solution (preferred pH 2 or less) of an inorganic acid such as hydrochloric acid, phosphoric acid or sulfuric acid, or an organic acid such as lactic acid, citric acid, acetic acid or an ascorbic acid, and then heated (preferably at 50 to 100° C.) while stirring for a certain time.

An alkali treatment may be carried out for example by stirring the cell residues obtained by enzymatic treatment or by other methods as above for several minutes to several hours in an alkaline aqueous solution having preferably a pH of 9 to 13, and more preferably from 10 to 12. The temperature of this aqueous solution is not restricted in any particular way but ranges ordinarily from 20 to 100° C., preferably from 30 to 100° C., and yet more preferably from 50 to 80° C. Compounds used for preparing the alkaline aqueous solution include for example aqueous solutions of inorganic salts such as sodium hydroxide, calcium hydroxide, potassium hydroxide, sodium silicate, etc.; and organic nitrogen compounds such as ammonia, monoethanoldiamine, ethylenediamine, diethylenetriamine, etc.

In an alcohol treatment, for example, alcohols such as monohydric alcohols, etc., or hydroalcohols may be added to the cell residues obtained by enzymatic treatment or by other methods as above and stirred for several minutes to several hours. The temperature of the treatment solution is not particularly restricted provided the alcohol does not evaporate, and ranges ordinarily from 20 to 80° C. The temperature ranges herein preferably from 30 to 60° C. More preferred methods involve for example stirring while heating at 40 to 50° C. Monohydric alcohols include for example lower alcohols having 1 to 6 carbon atoms such as methanol, ethanol, propanol, isopropyl alcohol, butanol, hexanol, etc., preferably ethanol.

The foreign substance encapsulated in the microorganism cell is liquid. Examples thereof include, but not limited to, a hydrophilic, hydrophobic or an amphiphilic liquid. The foreign substance is preferably a hydrophobic substance. A foreign substance herein refers to a substance artificially added to the microorganism cell to be encapsulated therein. This includes for example substances not derived from the microorganism components (endogenous components) as well as substances derived from the microorganism, substances extracted from the microorganism and then concentrated, mixed with other components or subjected to other kinds of artificial treatment. Placing these substances back into the cell counts herein as an artificial treatment; these substances are thus regarded as foreign substances according to the present invention.

Examples of the former include for example food additives, food compositions, pharmaceutical compositions, pharmaceutical additives, quasi-pharmaceutical compositions, cosmetic compositions, etc. Examples of the latter include for example microbial extracts such as yeast extracts, or bioactive substances derived from (or produced by) microorganism cells such as antibiotics or immunosuppressive pharmaceuticals, etc. Preferred herein are edible compositions such as food additives, food compositions (including food raw materials), oral pharmaceutical compositions, and oral pharmaceutical additives.

Specific examples of foreign substances include for example flavorings (including flavor components), colorings (including colorants, pigments and dyestuffs), sweeteners (including saccharides), acidulants, seasonings, bitterants, spices, fats/lipids, fatty acids, amino acids, enzymes, vitamins, animal or plant extracts, bioactive substances, etc. Preferred herein are components that can form flavor or taste (flavor/taste components) such as flavorings, sweeteners, acidulants, seasonings, bitterants, spices, fats/lipids, amino acids, vitamins, or, animal or plant extracts, etc.; colorings, vitamins, bioactive substances, etc. Yet more preferred herein are flavor/taste components, in particular flavorings, sweeteners, acidulants, seasonings, bitterants and spices.

These foreign substances may be foreign substances (active ingredients) by themselves having their intrinsic hydrophobic, hydrophilic or amphoteric character, preferably hydrophobic, or preparations wherein the foreign substances are heated or mixed with other substances such as solvents, preferably hydrophobic preparations.

Flavorings include herein ordinary flavorings widely used in the food, pharmaceutical and cosmetic industries, preferably flavorings suitable to be used in edible products such as foods, etc. Specific examples include for example citrus flavors such as orange, lemon, lime, grapefruit, mandarin, tangerine, etc.; other fruit flavors such as apple, banana, cherry, grape, melon, peach, pineapple, plum, raspberry, strawberry, etc.; bean flavors such as vanilla, coffee, cocoa, chocolate, etc.; mint flavors such as peppermint, spearmint etc.; spice flavors such as allspice, cinnamon, nutmeg, etc., nut flavors such as almond, peanut, walnut, etc.; aquatic product flavors such as crab, shrimp, fish, shellfish, etc.; and various kinds of other flavors from vegetables, cereals, seaweeds, etc. Flavor components include for example menthol, dl-menthol, menthone, vanillin, ethyl vanillin, cinnamic acid piperonal, d-borneol, maltol, ethyl maltol, camphor, methyl anthranilate, methyl cinnamate, cinnamic alcohol, methyl-N-methyl anthranilate, methyl β-naphthyl ketone, limonene, linalool, allyl isothiocyanate, etc. These may be used singly or in combinations of 2 or more.

Natural colors and synthetic colors may be used as colorings. Preferred herein are colorings suitable to be used in edible products such as food, etc. Examples of natural colors include carotenoid-based colors such as carotenoid colors, paprika color, annatto color, orange color, tomato color, marigold color, etc.; anthocyanin colors such as purple sweet potato color, red cabbage color, elderberry color, grape juice color, grape skin color, purple corn color, red radish color, perilla color, red kernel rice color, cowberry color, gooseberry color, cranberry color, salmonberry color, thimbleberry color, strawberry color, dark sweet cherry color, cherry color, hibiscus color, huckleberry color, black currant color, blackberry color, blueberry color, plum color, whortleberry color, boysenberry color, mulberry color, purple yam color, raspberry color, red currant color, loganberry color, etc.; quinoid-based colors such as cochineal color, Shikon color, madder color, Lac color, etc.; flavonoid-based colors such as cacao color, kooroo color, kaoliang color, Sandalwood red color, onion color, tamarind color, persimmon color, carob germ color, licorice color, Sappan wood color, peanut color, pecan nut color, Carthamus red color, Carthamus yellow color, etc.; azaphilone-based colors such as Monascus color, Monascus red color, etc.; and other colors such as Monascus yellow color, caramel, turmenic color, Kusagi color, Gardenia blue color; Gardenia yellow color, Gardenia red color, chlorophyllin color, chlorophyll, spirulina blue color, etc. Synthetic coal tar colors include for example Food Red No.2 (Amaranth), Food Red No.3 (Erythrosine), Food Red No.40 (Allura Red AC), Food Red No.102 (New Coccine), Food Red No.104 (Phloxine B), Food Red No.105 (Rose Bengale), Food Red No.106 (Acid Red), Food Yellow No.4 (Tartrazine), Food Yellow No.5 (Sunset Yellow FCF), Food Blue No.1 (Brilliant Blue FCF), Food Blue No.2 (Indigo Carmine), Food Green No.3 (Fast Green FCF), etc. Natural color derivatives include for example sodium norbixinate, potassium norbixinate, copper chlorophyll, sodium copper chlorophyllin and sodium iron chlorophyllin, etc.; synthetic natural colors include for example β-carotene, lutein, astaxanthin, canthaxanthin, riboflavin, riboflavin acetate, 5′-riboflavin phosphate ester sodium, etc. Preferred herein are β-carotene, carotenoid colors, paprika color, annatto color, madder color, orange color, Gardenia colors (Gardenia blue color; Gardenia yellow color, Gardenia red color), chlorophyll, Shikon color, Food Red No.3 (Erythrosine), Food Yellow No.4 (Tartrazine), onion color, tomato color, marigold color, lutein color, etc.

Sweeteners (including saccharides) include herein for example monosaccharides, disaccharides, oligosaccharides, sugar alcohols and high-intensity sweeteners. Specific examples thereof include for example monosaccharides such as arabinose, galactose, xylose, glucose, sorbose, fructose, rhamnose, ribose, isomerized liquid sugar (high fructose corn syrup), N-acetyl glucosamine, etc.; disaccharides such as isotrehalose, sucrose, trehalulose, trehalose, neotrehalose, palatinose, maltose, melibiose, lactulose, lactose, etc.; oligosaccharides such as α-cyclodextrin, β-cyclodextrin, isomalto oligosaccharides (isomaltose, isomaltotriose, panose, etc.), oligo-N-acetyl glucosamine, galactosyl sucrose, galactosyl lactose, galactopyranosyl (β1-3) galactopyranosyl (β1-4) glucopyranose, galactopyranosyl (β1-3) glucopyranose, galactopyranosyl (β1-6) galactopyranosyl (β1-4) glucopyranose, galactopyranosyl (β1-6) glucopyranose, xylooligosaccharides (xylotriose, xylobiose, etc.), gentioligosaccharides (gentiobiose, gentiotriose, gentiotetraose, etc.), stachyose, Theander oligosaccharides, nigero-oligosaccharides (nigerose, etc.), palatinose oligosaccharides, palatinose syrups, fucose, fructooligosaccharides (kestose, nistose, etc.), fructofuranosyl nistose, polydextrose, maltosyl β-cyclodextrin, maltooligosaccharides (maltotriose, tetraose, pentaose, hexaose, heptaose, etc.), raffinose, glycosyl sucrose syrups (coupling sugars), soybean oligosaccharides, invert sugars, glucose syrups, etc.; sugar alcohols such as isomaltitol, erythritol, xylitol, glycerol, sorbitol, palatinit, maltitol, maltoteraitol, maltotriitol, mannitol, lactitol, reduced isomalto-oligosaccharides, reduced xylooligosaccharides, reduced gentioligosaccharides, reduced maltose syrup, reduced glucose syrups, etc.; and high-intensity sweeteners such as α-glucosyltransferase treated stevia, aspartame, acesulfame potassium, alitame, liquorice extract (glycyrrhizin), triammonium glycyrrhizate, tripotassium glycyrrhizate, trisodium glycyrrhizate, diammonium glycyrrhizate, dipotassium glycyrrhizate, disodium glycyrrhizate, curculin, saccharine, saccharine sodium, cyclamate, sucralose, stevia extract, stevia powder, dulcin, thaumatin (somatin), tenryocha (Chinese blackberry tea) extract, serendipity berry extract, neotame, neohesperidine dihydrochalcone, fructosyltransferase treated stevia, Brazilian licorice extract, miracle fruit extract, luohan fruit extract, enzyme-treated liquorice, enzyme-decomposed liquorice, etc., and others sweeteners such as honey, fruit juices fruit juice concentrates, etc.

Fats/lipids include herein for example soybean oil, corn oil, rice bran oil, sunflower oil, cottonseed oil, olive oil, castor oil, fish oil, lard, beef tallow, sheep tallow, horse fat, lecithin, etc.

Fatty acids include for example docosahexaenoic acid, eicosapentaenoic acid, linoleic acid, linolenic acid, palmitic acid, oleic acid, etc.

Vitamins include for example vitamin A, B-group vitamins (vitamins B₁, B₂, B₆, B₁₂, B₁₃, B_{c}, Bₜ, etc.), vitamin C, vitamin D, vitamin E, vitamin H, vitamin K, vitamin M, vitamin P, vitamin U, etc., and their derivatives.

Animal or vegetable extracts include herein ordinary animal or vegetable extracts widely used in the food, pharmaceutical and cosmetic industries, preferably those suitable for use in edible products such as foods, etc. Examples thereof include for example vegetable extracts such as wheat extract, vanilla extract, quillaja extract, aloe vera extract, amacha extract, wheat germ extract, udo extract, gum benzoin extract, Aloe arborescens extract, konjac extract, eucalyptus leaf extract, cinchona extract, sour orange extract, Amur cork tree extract, Gentiana extract, grape seed extract, tea extract, etc.; and animal extracts such as beef extract, chicken extract, pork extract, crab extract, bonito extract, oyster extract, gelatin, collagen, yeast extract, etc. Animal or vegetable extracts include herein extracts of processed animals or vegetables such as extracts of dried shaved bonito flakes, etc., and animal or vegetable extracts subjected to further processing such as gelatin hydrolysates.

Bioactive substances include herein ordinary bioactive substances widely used in the food, pharmaceutical and cosmetic industries, preferably those suitable for use in edible products such as foods, oral pharmaceuticals, etc. Examples thereof include for example ethenzamide, sodium salicylate, acetoaminophen, ibuprofen, phenacetin, eugenol, antipyrine, catechins, saponins, chondroitin sulfate, phospholipids such as ceramides, etc.

Spices include herein ordinary spices widely used in the food, pharmaceutical and cosmetic industries. Preferred herein are those suitable for use in edible products such as foods, oral pharmaceuticals, etc. Examples thereof include, but not limited to, garlic, onion, lemongrass, chervil, tarragon, laurel, rosemary, basil, chili pepper, thyme, cinnamon, cashew, oregano, turmeric, clove, pepper, coriander, etc.

The components in the aforementioned groups belonging to the same category (i.e. groups of colorings, sweeteners, etc.) may be used singly or in combination of 2 or more.

Each of the aforementioned foreign substance groups may also be used singly or in combination of 2 or more. To the aforementioned foreign substances may also be added dispersants, antioxidants, preservatives, flavor improvers, emulsifiers, reinforcing agents, etc. For example, at least one antioxidant selected from the group consisting of extracted tocopherol, dl-α-tocopherol, d-α-tocopherol, dl-γ-tocopherol, mixed tocopherol, rosemary extract, Japanese bayberry extract, rutin extract, enzymatic hydrolysates of rutin, tea extract and tocotrienol may be used simultaneously with to the foreign substances encapsulated in the microorganism cells, in particular flavor/taste components, preferably flavorings.

The aforementioned foreign substances may be encapsulated inside the aforementioned microorganism cells by mixing the microorganism cells (cell bodies) with the foreign substances. Specifically, the foreign substances may be encapsulated inside the microorganism cells by adding the foreign substance to a liquid dispersion of microorganism cells (cell bodies), adjusting pH and temperature to desired values, and stirring as needed for a predetermined period of time. The pH is not particularly restricted herein and may be suitable selected ordinarily from within the range from 5 to 9, and preferably from 6 to 8. Temperature values are not particularly restricted and may be suitably selected so as to range ordinarily from 40 to 80° C., and preferably from 50 to 70° C.

There is no particular restriction as to the necessity of stirring, either. If any stirring equipment is used for stirring, for example blenders with stirring blades, emulsifying devices, dispersing devices, homogenizers, etc., the encapsulation of the foreign substance inside the microorganism cells can be carried out more effectively. The stirring speed or stirring rotation number per minute is not particularly restricted herein and may be suitable selected so as to range usually from 1,000 to 10,000 rpm.

When mixing the foreign substance with the aforementioned microorganism cells (cell bodies), other constituents may be further included in the mixture system of foreign substance and microorganism cells, for example hardeners, antioxidants, stabilizers, dispersants, emulsifiers, pH adjustment agents, preservatives, anti-denaturation agents, etc.

The present invention is further characterized in that at least one member among saccharides, high-intensity sweeteners, proteins and polyhydric alcohols is adhered onto the surface of the microcapsules (foreign-substance encapsulating microorganism cells) thus obtained by encapsulating foreign substances inside microorganism cells.

Examples of saccharides include for example monosaccharides such as arabinose, galactose, xylose, glucose, sorbose, fructose, rhamnose, ribose, isomerized liquid sugar (high fructose corn syrup), N-acetyl glucosamine, etc.; disaccharides such as isotrehalose, sucrose, trehalulose, trehalose, neotrehalose, palatinose, maltose, melibiose, lactulose, lactose, etc.; trisaccharides such as maltotriose, etc.; tetrasaccharides such as maltotetraose, etc.; oligosaccharides and polysaccharides such as α-cyclodextrin, β-cyclodextrin, isomalto oligosaccharides (isomaltose, isomaltotriose, panose, etc.), oligo-N-acetyl glucosamine, galactosyl sucrose, galactosyl lactose, galactopyranosyl (β1-3) galactopyranosyl (β1-4) glucopyranose, galactopyranosyl (β1-3) glucopyranose, galactopyranosyl (β1-6) galactopyranosyl (β1-4) glucopyranose, galactopyranosyl (β1-6) glucopyranose, xylooligosaccharides (xylotriose, xylobiose, etc.), gentioligosaccharides (gentiobiose, gentiotriose, gentiotetraose, etc.), stachyose, theander oligosaccharides, nigero-oligosaccharides (nigerose, etc.), palatinose oligosaccharides, palatinose syrups, fucose, fructooligosaccharides (kestose, nistose, etc.), fructofuranosyl nistose, polydextrose, maltosyl β-cyclodextrin, maltooligosaccharides (maltotriose, tetraose, pentaose, hexaose, heptaose, etc.), raffinose, glycosyl sucrose syrups (coupling sugars), soybean oligosaccharides, invert sugars, glucose syrups, dextrin, gum arabic, xanthan gum, guar gum, carrageenan, curdlan, tamarind seed gum, gellan gum, starches, modified starches, etc.; sugar alcohols such as isomaltitol, erythritol, xylitol, glycerol, sorbitol, palatinit, maltitol, maltoteraitol, maltotriitol, mannitol, lactitol, reduced isomalto-oligosaccharides, reduced xylooligosaccharides, reduced gentioligosaccharides, reduced maltose syrup, reduced glucose syrups, etc. Preferred herein are monosaccharides such as glucose, ribose, arabinose, xylose, mannose, galactose, fructose, etc.; disaccharides such as maltose, lactose, sucrose, trehalose, etc.; trisaccharides such as maltotriose, etc.; tetrasaccharides such as maltotetraose, etc.; oligosaccharides such as maltooligosaccharides, etc.; polysaccharides such as dextrin, gum arabic, xanthan gum, guar gum, gellan gum, starches, modified starches, cyclodextrin, etc.; sugar alcohols such as palatinit, sorbit, xylit, maltitol, lactitol, erythritol. Yet more preferred are disaccharides such as lactose, trehalose, etc. Such saccharides may be used singly or in combination of 2 or more.

High-intensity sweeteners include for example α-glucosyltransferase treated stevia, aspartame, acesulfame potassium, alitame, liquorice extract (glycyrrhizin), triammonium glycyrrhizate, tripotassium glycyrrhizate, trisodium glycyrrhizate, diammonium glycyrrhizate, dipotassium glycyrrhizate, disodium glycyrrhizate, curculin, saccharine, saccharine sodium, cyclamate, sucralose, stevia extract, stevia powder, dulcin, thaumatin (somatin), tenryocha (Chinese blackberry tea) extract, Serendipity berry extract, neotame, neohesperidine dihydrochalcone, fructosyltransferase treated stevia, Brazilian licorice extract, miracle fruit extract, luohan fruit extract, enzyme-treated liquorice, enzyme-decomposed liquorice, etc., preferably aspartame, sucralose, acesulfame potassium, somatin, stevia, alitame, neotame and xylitol. These high-intensity sweeteners may be used singly or in combination of 2 or more.

Proteins include for example gelatin, gelatin hydrolysates, maize proteins, casein, sodium caseinate, collagen, etc. They include preferably gelatin, maize proteins and sodium caseinate. These proteins may be used singly or in combination of 2 or more.

Polyhydric alcohols include for example glycerin, propylene glycol, and polymers thereof such as polypropylene glycol, polyglycerin, etc. Propylene glycol and glycerin are preferred. These polyhydric alcohols may be used singly or in combination of 2 or more.

Furthermore, these saccharides, high-intensity sweeteners, proteins and polyhydric alcohols may also be used singly or in combination of 2 or more.

The methods for adhering saccharides, high-intensity sweeteners, proteins and polyhydric alcohols (hereinafter referred to as ‘saccharides, etc.’) onto the aforementioned microcapsules are not restricted in any particular way. They may include for example methods wherein the microcapsules (foreign-substance encapsulating microorganism cells) prepared according to a method described above are dried in a spray-drying machine and are then sprayed using a fluidized bed granulator, etc. with solutions or dispersions of ‘saccharides, etc.’ dissolved or dispersed in water or other solvent; methods wherein a bed spread over with the ‘saccharides, etc.’ (fluid bed) is sprayed with a dispersion of microcapsules (foreign-substance encapsulating microorganism cells), and then the droplets of microcapsules with their surface covered with ‘saccharides, etc.’ are taken out and dried; methods wherein the ‘saccharides, etc.’ are added to a liquid dispersion of the microcapsules (foreign-substance encapsulating microorganism cells), are homogeneously dissolved therein and are then lyophilized; methods wherein the microcapsules (foreign-substance encapsulating microorganism cells) are dried by spray-drying, etc. and the ‘saccharides, etc.’ are mixed therewith as a powder mixture using a high speed stirring mixer, etc.; and methods wherein the ‘saccharides, etc.’ are added to a liquid dispersion of the microcapsules (foreign-substance encapsulating microorganism cells), are homogeneously dissolved therein and this liquid dispersion is then spray-dried, etc. Preferred methods are those wherein the ‘saccharides, etc.’ are added to a liquid dispersion of the microcapsules (foreign-substance encapsulating microorganism cells), are homogeneously dissolved therein and this liquid dispersion is then spray-dried.

The proportion of the ‘saccharides, etc.’ (saccharides, high-intensity sweeteners, proteins or polyhydric alcohols) relative to 100 wt % of the above microcapsules (foreign-substance encapsulating microorganism cells) usually ranges from 1 to 90 wt %, preferably from 10 to 50 wt %, and yet more preferably from 20 to 40 wt % (dried solids weight ratios).

In the above method, if the microcapsules (foreign-substance encapsulating microorganism cells) are used as a dispersion/suspension in a liquid solution, the proportion of microcapsules in these liquid dispersion/suspension usually ranges from 5 to 80 wt %, preferably from 10 to 50 wt %, yet more preferably from 20 to 40 wt %; if the ‘saccharides, etc.’ are also used as a liquid solution or dispersion/suspension, their proportion in these solutions usually ranges from 1 to 60 wt %, preferably from 10 to 50 wt %, and yet more preferably from 30 to 40 wt %.

The microcapsules thus obtained have ‘saccharides, etc.’ (saccharides, high-intensity sweeteners, proteins or polyhydric alcohols) adhered at least partially onto their surfaces. Herein, the surface of the microcapsules is preferably caused to be covered (coated) partially or completely with ‘saccharides, etc.’. The ‘saccharides, etc.’ may also permeate/penetrate into the microcapsule film (cell wall) or into the interior of the microcapsule, so long as some ‘saccharides, etc.’ remain adhered at least partially to the surface of the microcapsule.

Such microcapsules according to the present invention, i.e. microcapsules with ‘saccharides, etc.’ adhered to their surfaces or, preferably, having their surfaces covered with ‘saccharides, etc.’, may be used as components in foods, pharmaceuticals, quasi-pharmaceuticals, cosmetics and feeds. The microcapsules according to the present invention may be employed preferably as components in oral compositions used orally (food compositions, oral pharmaceutical or quasi-pharmaceuticals compositions, and feed compositions). Oral compositions include herein physiologically ingestible edible compositions as well as buccal compositions, edible or not, for use in the mouth.

The microcapsules according to the present invention (microcapsules with adhered (coating) ‘saccharides, etc.’) may have different applications depending on the kind of the encapsulated components. For example, microcapsules enclosing flavorings may be prepared and used as flavorings (microencapsulated flavorings); microcapsules enclosing sweeteners, as sweeteners (microencapsulated sweeteners); microcapsules enclosing seasonings, as seasonings (microencapsulated seasonings); microcapsules enclosing acidulants, as acidulants (microencapsulated acidulants); microcapsules enclosing bitterants, as bitterants (microencapsulated bitterants); microcapsules enclosing spices, as spices (microencapsulated spices); and microcapsules enclosing colorings, as colorants (microencapsulated colorants).

The microcapsules according to the present invention may be suitably used as a component in embodiments of oral compositions that are ingested while being held or chewed in the mouth. Specific examples of such oral compositions include for example chewing gum, soft candy such as gummy, nougat, etc., or food compositions held in the mouth for a prolonged time such as sweets, nonessential grocery foods etc.; pharmaceutical or quasi-pharmaceutical compositions that are ingested while being held or chewed in the mouth such as chewable tablets, masticatories, granules, troche tablets, buccal tablets, dentifrices, etc. Preferred herein are compositions ingested while being chewed for a prolonged time in the mouth such as chewing gum. In the present invention, the term chewing gum applies in a broad sense to any gums taken by chewing in the mouth including bubble gum, etc.

The present invention provides oral compositions containing the aforementioned microcapsules with adhered (coating) ‘saccharides, etc.’ (microcapsules with adhered (coating) ‘saccharides, etc.’: microcapsules according to the present invention). As follows from all the above, the oral compositions according to the present invention encompass thus compositions used in the mouth (buccal compositions) and compositions ingested (eaten) orally (edible compositions), such as food compositions, oral pharmaceuticals or quasi-pharmaceuticals compositions, and feed compositions.

Specific examples of such oral compositions are similar to those listed above, for example the aforementioned chewing gum, soft candy such as gummy, nougat, etc.; food compositions held in the mouth for a prolonged time such as sweets, nonessential grocery foods, etc.; pharmaceutical or quasi-pharmaceutical compositions that are taken while being held or chewed in the mouth for prolong time such as chewable tablets, masticatories, granules, troche tablets, buccal tablets, dentifrices, etc. Preferred herein are food compositions, and more preferably chewing gum.

Apart from containing as constituents with microcapsules with adhered (coating) ‘saccharides, etc.’ (microcapsules according to the present invention), the oral compositions according to the present invention, especially food compositions and in particular chewing gum may also contain constituents of conventional oral compositions, especially of food compositions and in particular of chewing gum, and may be manufactured using conventional methods.

In the case of a chewing gum, there is no restriction as to the amount of microcapsules according to the present invention (microcapsules with adhered (coating) ‘saccharides, etc.’) in 100 wt % of the final chewing gum, but it usually ranges from 0.05 to 10 wt %, preferably from 0.1 to 5 wt %, and yet more preferably from 0.5 to 3 wt %.

The microcapsules according to the present invention in oral compositions such as chewing gum, etc. are not particularly restricted concerning their intended use, but they contain inside preferably at least one of the following: flavorings, sweeteners (including saccharides), colorings, acidulants, vitamins, spices, animal/plant extracts, bioactive substances, seasonings, bitterants, fats/lipids, fatty acids, amino acids or enzymes. Preferred in case of a chewing gum are components for building flavor or taste (flavor/taste components), in particular flavorings, sweeteners, acidulants, seasonings or bitterants. Microcapsules (oral compositions) according to the present invention containing these flavorings, sweeteners, acidulants, seasonings or bitterants may be formulated respectively in a chewing gum (oral composition) as, respectively, microencapsulated flavorings, microencapsulated sweeteners, microencapsulated acidulants, microencapsulated seasonings and microencapsulated bitterants.

Specific examples of such foreign substance components (flavorings, sweeteners, colorings, etc.) include the same examples as listed above, but are not limited thereto. For example, flavorings other than the flavorings listed above may also be used as flavor component.

Apart from the microcapsules according to the present invention, the chewing gum formulation may include other components such as gum base, sweeteners (including saccharides), colorings, thickeners, acidulants, softeners, etc. The amount of gum base in the chewing gum ranges ordinarily from 10 to 35 wt %, and preferably from 20 to 30 wt %. Sweeteners herein include for example saccharides such as sucrose, fructose, liquid sugar, glucose, oligosaccharides, etc.; high-intensity sweeteners such as aspartame, sucralose, acesulfame potassium, somatin, stevia, alitame, neotame, xylitol, saccharine salts, glycyrrhizin, etc.

Chewing gums may be prepared for example in accordance with the methods and procedures described in “Gum Base and Gum Products Technology” and “The Great American Chewing Gum Book” by Robert Hendrickson, published by CAFOSA Gum S/A (1974). The constituent components of the microcapsules according to the present invention may be incorporated therein in any arbitrary process step, but preferably close to the end of the manufacturing process, especially in such cases where the foreign substances to be encapsulated in the microcapsules are sensitive to heat, for example flavor/taste components (flavorings in particular), colorings, etc., in order to prevent the thermal alternation of these foreign substances (i.e. loss or denaturation of flavor or color, etc.).

(2) Manufacturing Method for Microcapsules

The present invention relates further to a manufacturing method for the aforementioned microcapsules according to the present invention (microcapsules with adhered (coating) ‘saccharides, etc.’ or a cover thereof).

This method comprises essentially the following steps:
1) encapsulating a foreign substance inside microorganism cells;
2) adhering onto the surface of the microorganism cells obtained in step (1) at least one member selected from the group consisting of saccharides, high-intensity sweeteners, proteins and polyhydric alcohols.

Between steps 1) and 2) the microorganism cells encapsulating the foreign substance may be subject as needed to further treatments, such as separation, washing, dehydration, drying, etc.

Alternatively, microorganism cells with their endogenous intracellular component eluted beforehand may be used as the microorganism cells of step 1) above.

The microorganism cells, foreign substances, saccharides, high-intensity sweeteners, proteins and polyhydric alcohols used in the aforementioned manufacturing method may be those described in section (1) above, while the methods for eluting the endogenous intracellular component out of the microorganism cells, the methods for encapsulating the foreign substances in the microorganism cells, and the methods for adhering saccharides, high-intensity sweeteners, proteins and polyhydric alcohols onto the surface of the microorganism may be carried out also in accordance with the method described in section (1) above.

(3) Method for Controlling the Expression of Flavor/Taste or the Release of Flavor/Taste Components

The present invention also relates to a method for controlling the speed, intensity and persistence of the release of foreign substances in microcapsules of foreign substances encapsulated in microorganism cells (foreign-substance encapsulating microorganism cells), preferably for increasing the speed, intensity and persistence of the release of the foreign substances.

This method may be implemented essentially by adhering onto the surfaces of the microorganism cells that encapsulate foreign substances (foreign-substance encapsulating microorganism cells) at least one member selected from the group consisting of saccharides, high-intensity sweeteners, proteins and polyhydric alcohols. The microorganism cells used herein for encapsulating foreign substances in their cells may include the aforementioned algae, yeasts, fungi and bacteria, preferably yeasts. More preferably, yeasts with their endogenous intracellular components eluted beforehand.

Herein, the yeasts, foreign substances, saccharides, high-intensity sweeteners, proteins and polyhydric alcohols used in the aforementioned method may be those described in section (1) above, while the methods for eluting the endogenous intracellular component out of the microorganism cells, the methods for encapsulating the foreign substances in the microorganism cells, and the methods for adhering saccharides, high-intensity sweeteners, proteins and polyhydric alcohols onto the surface of the microorganism may be carried out also in accordance with the method described in section (1) above. Also, the extent to which the saccharides, high-intensity sweeteners, proteins and polyhydric alcohols (‘saccharides, etc.’) are adhered onto the surface of the microorganism cells encapsulating the foreign substances is not particularly restricted, so that the microorganism may be completely or partially covered. The ‘saccharides, etc.’ may also permeate/penetrate into the microorganism cell wall or inside the cell, without any particular restriction. The proportion of ‘saccharides, etc.’ adhered to (coating) the surface of the microorganism cells encapsulating the foreign substances, relative to 100 wt % of the foreign-substance encapsulating microorganism usually ranges from 1 to 90 wt %, preferably from 10 to 50 wt %, and yet more preferably from 20 to 40 wt % (dried solids weight ratios).

The microcapsules (microcapsules with adhered (coating) “saccharides, etc.) thus obtained allow the control of the speed, intensity and persistence of the release of the foreign substances encapsulated therein. This effect becomes more pronounced especially by placing the “microcapsules with adhered (coating) ‘saccharides, etc.’” in the presence of water, in particular by placing them inside the mouth. Therefore, the method according to the present invention may be suitably used for controlling the speed, intensity and persistence of the release of flavor/taste components inside the mouth.

Thus the present invention provides a suitable method for controlling the speed, intensity and persistence of the expression of flavor/taste, preferably for increasing the speed, intensity and persistence of the expression of flavor/taste, in microcapsules of a flavor/taste components encapsulated in microorganism cells (foreign-substance encapsulating microorganism cells).

Flavor/taste components herein include compounds for building flavor (smell) or taste. These include, but are not limited to, flavorings, sweeteners (including saccharides), acidulants, seasonings, bitterants, spices, fats/lipids, vitamins, amino acids, and animal and plant extracts, etc. More preferably, they include flavorings, sweeteners, acidulants, seasonings, bitterants and spices, yet more preferably flavorings and sweeteners, and more preferably still flavorings. These components are all preferably edible components suitable for use in foods. Yet more preferably, they are hydrophobic components such as hydrophobic flavorings, etc.

The “microcapsules with adhered (coating) ‘saccharides, etc.’” obtained by adhering (coating with) ‘saccharides, etc.’ onto the surfaces of microorganism cells encapsulating flavor/taste components (microcapsules) are more effective in achieving a faster expression of flavor/taste in the mouth and prolonging a moderate intensity of flavor/taste than is the case for unmodified microorganism cells encapsulating a flavor/taste components (microcapsule).

The method according to the present invention is therefore a method for controlling the release of flavor/taste components encapsulated in microcapsules (microorganism cells) from the microcapsules. Specifically, the method according to the present invention may be used as a technique for increasing the speed, intensity and persistence of the release or expression of flavor/taste components (flavorings, sweeteners, acidulants, seasonings, bitterants, spices, fats/lipids, vitamins, amino acids, or animal/plant extracts, etc.).

Furthermore, by including as flavor/taste components (flavorings, sweeteners, acidulants, seasonings, bitterants, spices, fats/lipids, vitamins, amino acids, or animal/plant extracts, etc.) the microcapsules according to the present invention used herein (microcapsules with adhered (coating) ‘saccharides, etc.’) in oral compositions such as food, etc., it becomes possible to control the speed, intensity and persistence of the expression of flavor/taste in such oral compositions.

Thus the present invention provides a suitable method for controlling the speed, intensity and persistence of the expression of flavor/taste, preferably for increasing the speed, intensity and persistence of the expression of flavor/taste, in oral compositions such as food, etc. Suitable oral compositions herein include compositions held or chewed for a prolonged time in the mouth, for example food compositions such as chewing gum, soft candy, etc.; pharmaceutical or quasi-pharmaceutical compositions such as chewable tablets, masticatories, buccal tablets, dentifrices, etc. A preferred oral composition herein is chewing gum. This method may be implemented by using as a component of the target oral composition the “microcapsules with adhered (coating) ‘saccharides, etc.’” above, i.e., by preparing the target oral composition using the “microcapsules with adhered (coating) ‘saccharides, etc.’” as one component thereof. The proportion of “microcapsules with adhered (coating) ‘saccharides, etc.’” used in the preparation the oral composition, in particular chewing gum, etc. may be those described in section (1) above, while the preparation method thereof may be carried out also in accordance with the method described in section (1) above.

### EXAMPLES

The present invention will be described below in detail with reference to the following Examples, Comparative Examples, and Experiments, but is not limited thereto. In each Example, unless otherwise specified, “part” means “part by weight” and “%” means “% by weight”.

### Examples 1 to 6

Saccharide-Adhered (Coated) Microcapsule Containing Flavorings (Intracellular Components Removed) (Microencapsulated Flavoring)

(1) Removal of Endogenous Intracellular Components from the Yeast Cell Body

2 g of protease was added to 2,000 g of slurry in which 100 g of a brewer's yeast (saccharomyces cerevisiae) was dispersed in water, and the resulting mixture was then shaken at 50° C. for 20 hours to elute the intracellular components from the interior of the cell outside. The obtained slurry was separated by centrifugation at 8,000 rpm for 30 minutes and the supernatant was removed, giving 350 g (solids content of 20%) of yeast residue. 930 g of water and 120 g of concentrated hydrochloric acid were added thereto and the resulting mixture was heated for 10 minutes at 80° C. while stirring (acid treatment), followed by cooling. After centrifuging at 8,000 rpm for 30 minutes, the supernatant was removed, giving 500 g (solids content of 10%) of acid-treated yeast residue. Subsequently, 500 g of water was added thereto, followed by centrifugation at 8,000 rpm for 30 minutes, and the supernatant was removed. 10% aqueous sodium hydroxide was added to 500 g of the obtained residue (solids content of 10%) and the resulting solution was adjusted to pH 7.0.

(2) Inclusion of Flavorings into Yeast Cell Bodies

450 g (solids content of 10%) of the above-prepared aqueous dispersion containing the yeast from which endogenous components had been removed from the yeast cell bodies was heated at 70° C., and 25 g of menthol was added thereto, followed by stirring at 5,000 rpm for 2 hours, whereby a flavoring (menthol) was included into the yeast cell bodies (flavoring-containing yeast).

(3) Adhesion (Coating) of Saccharides to the Surface of Yeast Cell Bodies

475 g of each of the thus prepared aqueous solutions containing the flavoring-containing yeast were prepared (6 types). To each aqueous solution was mixed either 60 g of an aqueous solution with a lactose content of 50% heated at 60° C. (Example 1), 60 g of an aqueous solution with a dextrin (DE=10) content of 50% (Example 2), 60 g of aqueous solution with a maltose content of 50% (Example 3), 60 g of an aqueous solution with reduced paratinose content of 50% (Example 4), 120 g of an aqueous solution with a gelatin content of 25% (Example 5), or 100 g of an aqueous solution containing 15% gelatin and 15% lactose (Example 6). Subsequently, these mixed solutions were spray-dried with a spray dryer at an inlet temperature of 150° C. and an outlet temperature of 90° C., to give 90 g each of powdered “saccharide-adhered microcapsule containing flavorings (intracellular components removed)” (Examples 1 to 6).

### Example 7

Saccharide-Adhered (Coated) Microcapsule Containing Flavorings (Intracellular Components Contained) (Microencapsulated Flavorings)

A procedure was conducted (using an aqueous solution with a lactose content of 50%) in the same manner as in Example 1 except that a brewer's yeast (saccharomyces cerevisiae) containing endogenous intracellular components was used in place of the yeast from which endogenous intracellular components had been removed from the cell body, to give a powdered saccharide-adhered microcapsule containing flavorings (intracellular components contained) (Example 7).

450 g of an aqueous solution containing 45 g of yeast (saccharomyces cerevisiae) was heated at 70° C. according to the method described in Example 1 (2), and 25 g of menthol was added thereto, followed by stirring at 5,000 r.p.m. for 2 hours, whereby a flavoring (menthol) was included in the yeast cell bodies (flavoring-containing yeast). To 475 g of an aqueous solution containing the thus-prepared flavoring-containing yeast was mixed 30 g of an aqueous solution with a lactose content of 50% heated at 60° C. according to the method described in the above-described Example 1 (3). Subsequently, the mixed solution was spray-dried with a spray dryer at an inlet temperature of 150° C. and an outlet temperature of 90° C., to prepare 90 g of powdered “saccharide-adhered microcapsule containing flavorings (intracellular components contained)” (Example 7).

### Examples 8 to 14

### Chewing Gum

The microcapsule containing flavor prepared in Examples 1 to 7 were used as a flavoring ingredient (microencapsulated flavoring) to prepare chewing gums according to the following formulation. Chewing gums were prepared in the usual manner.

<Formulation of chewing gum>

| | |
|---|---|
| Gum base | 25 parts |
| Sugar | 62 parts |
| Corn syrup | 10 parts |
| Glycerin | 1 part |
| Microencapsulated | 2 parts |
| flavoring | |
| Total | 100 parts |

### Example 15

Saccharide-Adhered (Coated) Microcapsule Containing Flavorings (Intracellular Components Removed) (Microencapsulated Flavorings)

450 g of a yeast solution (solids content of 10%) was prepared by dispersing in water yeast from which endogenous intracellular components had been removed from the cell body prepared in the same manner as in Example 1 (1). The solution was heated at 70° C., and 25 g of orange oil was added thereto, followed by stirring at 5,000 rpm for 2 hours. 60 g of an aqueous solution with lactose content of 50% was added thereto and mixed. Subsequently, the mixed solution was spray-dried with a spray dryer at an inlet temperature of 150° C. and an outlet temperature of 90° C., to obtain 90 g of a powdered “saccharide-adhered microcapsule containing flavorings (intracellular components removed)” (Example 15).

### Comparative Example 1

### Microcapsule Containing Flavorings (Intracellular Components Removed)

643 g of a yeast solution (solids content of 10%) was prepared by dispersing in water yeast from which endogenous intracellular components had been removed from the cell body prepared in the same manner as in Example 1 (1). The solution was heated at 70° C., and 36 g of menthol was added thereto, followed by stirring at 5,000 rpm for 2 hours. Subsequently, the solution was spray-dried with a spray dryer at an inlet temperature of 150° C. and an outlet temperature of 90° C., to obtain 90 g of a powdered microcapsule (microcapsule containing flavorings (intracellular components removed)) (Comparative Example 1) .

### Comparative Example 2

### Mixed Composition of a Microcapsule Containing Flavorings (Intracellular Components Removed) and Lactose

30 g of powdered lactose was added and mixed to 70 g of powdered microcapsules (microcapsule containing flavorings (intracellular components removed)) prepared in Comparative Example 1, giving 98 g of a powdered microcapsule content composite (flavoring-encapsulated microcapsule (intracellular components removed) and lactose) (Comparative Example 2).

### Comparative Example 3

### Microcapsule Containing Flavorings (Intracellular Components Contained)

750 g of a yeast solution (solids content of 10%) was prepared by dispersing 75 g of brewer's yeast (saccharomyces cerevisiae) in water. The resulting solution was heated at 70° C., and 25 g of menthol was added thereto, followed by stirring at 5,000 rpm for 2 hours. Subsequently, the solution was spray-dried with a spray dryer at an inlet temperature of 150° C. and an outlet temperature of 90° C., to prepare 90 g of powdered microcapsule (microcapsule containing flavorings (intracellular components containing)) (Comparative Example 3).

### Comparative Examples 4 to 6

### Chewing Gum

Each of the microcapsule examples prepared in Comparative Examples 1 to 3 was used as a flavoring ingredient (microencapsulated flavorings) to prepare chewing gums (Comparative Examples 4 to 6) in the same manner as in the above Examples 8 to 14.

### Comparative Example 7

### Microcapsule Containing Flavorings (Intracellular Components Removed)

750 g of a yeast solution (solids content of 10%) was prepared by dispersing in water yeast from which endogenous intracellular components had been removed from the cell body prepared in the same manner as in Example 1 (1). The solution was heated at 70° C., and 25 g of orange oil was added thereto, followed by stirring at 5,000 rpm for 2 hours. Subsequently, the solution was spray-dried with a spray dryer at an inlet temperature of 150° C. and an outlet temperature of 90° C., to prepare 90 g of powdered microcapsule (microcapsule containing flavorings (intracellular components removed)) (Comparative Example 7).

### Comparative Example 8

### Microencapsulated Flavors (Non-Microorganism)

250 g of a solution (solids content of 30%) prepared by dissolving 30 g of gum arabic and 30 g of lactose in water was heated to 50° C., and 25 g of orange oil was added thereto, followed by stirring at 5,000 rpm for 2 hours. Subsequently, the resulting solution was spray-dried with a spray dryer at an inlet temperature of 150° C. and an outlet temperature of 90° C., to obtain 90 g of a powdered microencapsulated flavorings.

### Experimental Example 1

Expression speed and persistence of the flavor/taste in the microcapsule containing flavorings (microcapsulated flavorings) prepared in Example 1 and Comparative Example 2 were evaluated. (Example 1: lactose-adhered microcapsule containing flavorings (intracellular components removed), Comparative Example 1: microcapsule containing flavor (intracellular components removed)+lactose). Specifically, six expert panelists chewed 75 times per minute the chewing gum (Example 8 and Comparative Example 5) comprising the microcapsulated flavorings (Example 1 and Comparative Example 2) as the flavoring ingredient. The speed of the expression, intensity and persistence of the flavor/taste in the mouse were evaluated compared to those of chewing gum (Comparative Example 4) prepared using the microcapsule (microcapsulated flavorings: flavorings-containing microcapsule (intracellular components removed)) of Comparative Example 1 as a flavoring ingredient, which was defined as the control. The evaluation was conducted based on the criteria shown in Table 1 with the expression speed, intensity and persistence of flavor/taste of the control-chewing gum (Comparative Example 4) being defined as 3.

TABLE 1

| | Expression | Intensity | Persistence |
|---|---|---|---|
| Evaluation | speed of the | of the | of the |
| scale | flavor/taste | flavor/taste | flavor/taste |
| 5 | Fast | Strong | Long |
| 4 | Relatively | Relatively | Relatively |
| | fast | strong | long |
| 3 | Average | Average | Average |
| (Control) | | | |
| 2 | Relatively | Relatively | Relatively |
| | slow | weak | short |
| 1 | Slow | Weak | Short |

Table 2 shows the results. The scores are expressed as the average of those of the six expert panelists.

TABLE 2

| | Types | | Expression | Intensity | |
|---|---|---|---|---|---|
| | of | State of | speed of | of the | Persistence |
| | saccha- | saccha- | the | flavor/ | of the |
| | ride | ride | flavor/taste | taste | flavor/taste |
| Examples | Lactose | Adhered | 4.8 | 4.8 | 4.8 |
| 1/8 | | | | | |
| Compar- | Lactose | Mixed | 3.0 | 3.4 | 3.0 |
| ative | | | | | |
| Examples | | | | | |
| 2/5 | | | | | |

In the table, “Examples 1/8” and “Comparative Examples 2/5” respectively indicate Examples 1 and 8, and Comparative Examples 2 and 5. The tables below follow the same rule.

As is clear from the results of the test conducted using the chewing gum of Example 8, it is possible to prepare chewing gum having a fast expression, intense and persistent of flavor/taste inside the mouth by using a microcapsulated flavoring (Example 1) comprising a flavoring-containing yeast (intracellular components removed) covered with lactose as a flavoring ingredient. Chewing gum (Comparative Example 5) prepared by using a microcapsulated flavoring (Comparative Example 2) obtained by merely mixing a flavoring-containing yeast (intracellular components removed) and lactose as a flavoring ingredient was evaluated almost the same as the control gum (speed of expression, intensity and persistence of flavor/taste in the mouth: 3.0) in which the speed of expression, intensity and persistence of flavor/taste are significantly inferior to the gum of Example 8 of the present invention. These results made it clear that by adhering to or covering the surface of the flavor-containing yeast with lactose, the distinctive effects of the present invention can be achieved, and these cannot be attained by merely mixing the yeast with lactose.

### Experimental Example 2

Expression speed, intensity and persistence of flavor/taste were evaluated in the same manner as in Experimental Example 1, using chewing gum (Example 12 and Comparative Example 6) prepared using microcapsulated flavorings of Example 7 and Comparative Example 3 (Example 7: lactose-adhered microcapsule containing flavorings (intracellular components containing), Comparative Example 3: flavoring-encapsulated microcapsule (intracellular components containing)) as a flavoring ingredient without removing the endogenous intracellular components from yeast cell body. Table 3 shows the results.

TABLE 3

| | | | | | Persis- |
|---|---|---|---|---|---|
| | | | Expression | Intensity | tence |
| | Presence of | Presence | speed of | of the | of the |
| | intracellular | of | the | flavor/ | flavor/ |
| | components | saccharide | flavor/taste | taste | taste |
| Examples | Present | Lactose | 4.8 | 3.6 | 3.6 |
| 7/14 | | | | | |
| Comp. | Present | Not | 3.0 | 3.0 | 3.0 |
| Examples | | present | | | |
| 3/6 | | | | | |

As is clear from these results, the chewing gum (Example 14) comprising the microcapsulated flavorings (Example 7: lactose-adhered microcapsule containing flavorings (intracellular components containing)) that was prepared by enclosing the flavorings in yeast without removing the endogenous intracellular component from the yeast cell body and adhering lactose on the surface thereof was significantly superior to chewing gum (Comparative Example 6) comprising the microcapsulated flavorings (Comparative Example 3: flavoring-encapsulated microcapsule (intracellular components containing)) obtained without adhering saccharide on the surface of the cell body in the expression speed, intensity and persistence of flavorings.

### Experimental Example 3

Expression (speed and intensity) and persistence of flavor were evaluated in the same manner as in Experimental Example 1 using chewing gum (Examples 9 to 11) prepared in the same manner as Example 8 except that the microcapsulated flavorings obtained in Example 2-to 4 (Example 2: dextrin-adhered microcapsule containing flavorings (intracellular components removed), Example 3: maltose-adhered microcapsule containing flavorings (intracellular components removed), Example 4: reduced palatinit-adhered microcapsule containing flavorings (intracellular components removed)) instead of that obtained in Example 1 (lactose-adhered microcapsule containing flavorings (intracellular components removed)).

The results are shown in table 4 together with the test results obtained using the chewing gum (using lactose as the adhered saccharide) of Example 8.

TABLE 4

| | Types of | Expression | Intensity | Persistence |
|---|---|---|---|---|
| | the adhered | speed of the | of the | of the |
| | saccharide | flavor/taste | flavor/taste | flavor/taste |
| Examples | Lactose | 4.8 | 4.8 | 4.8 |
| 1/8 | | | | |
| Examples | Dextrin | 4.2 | 3.0 | 4.8 |
| 2/9 | (DE10) | | | |
| Examples | Maltose | 2.4 | 2.8 | 4.8 |
| 3/10 | | | | |
| Examples | Reduced | 5.0 | 4.0 | 3.6 |
| 4/11 | palatinit | | | |
| Examples | Gelatin | 3.0 | 2.0 | 5.0 |
| 5/12 | | | | |
| Examples | Gelatin + | 4.0 | 2.5 | 4.9 |
| 6/13 | Lactose | | | |

As is clear from the results of Examples 9 and 10, the microcapsulated flavorings having the surfaces covered with dextrin (DE10) or maltose exhibited an intensity of the flavor/taste almost the same as that of the control (Comparative Example 4); however, the persistence of flavor/taste was significantly improved. As is clear form the results of Example 11, the microcapsulated flavorings with their surface covered by reduced palatinit remarkably improved the intensity of the flavor/taste.

Furthermore, the results of Examples 12 and 13 show that microcapsulated flavorings having their surfaces covered with gelatin or gelatin+lactose exhibited a flavor intensity weaker than that of the control (Comparative Example 4); however, the persistence of the flavor/taste thereof was significantly improved.

### Experimental Example 4

Microcapsules of Example 15 and Comparative Examples 7 and 8 (Example 15: saccharide-adhered microcapsule containing flavorings (intracellular components removed), Comparative Example 7: flavoring-encapsulated microcapsule (intracellular components removed), Comparative Example 8: microcapsulated flavorings (non-microbial)) were evaluated for the intensity and persistence of flavor/tasted.

Specifically, 50 g of each microcapsule was put into a plastic bag, sealed, and stored at 50° C. for a month. To evaluate the persistence of flavor/taste enclosed in the microcapsules, the content of limonene (orange flavor) in the microcapsules stored for a month was measured and the remaining ratio thereof was calculated, having defined the content of limonene before storage as 100%. The microcapsules (before storage) of Example 15 and Comparative Examples 7 and 8 contained 23 % limonene based on the weight of the microcapsule (100%).

To examine the change in quality of the flavorings after storage, 0.1% solutions were prepared using the microcapsules before and after storage at 50° C. for a month, and six expert panelists evaluated the flavor. Table 5 shows the results.

TABLE 5

| | Remaining | |
|---|---|---|
| | amount of | Flavoring of 0.1% |
| | limonene | solution |
| Example 15 | 95% | No change |
| Comp. Example 7 | 70% | Slightly oxidized odor |
| Comp. Example 8 | 45% | Strongly oxidized odor |

From these results, it became clear that persistence of flavor can be improved by covering the surface of the flavoring-encapsulated yeast (microcapsule) with saccharide (Example 15), and changes in quality of the flavor can be significantly prevented.

### Industrial Applicability

The present invention can improve the release speed, intensity and persistence of foreign materials contained in microorganism cells (microcapsules). The microcapsules having a controlled and/or improved speed, intensity and/or persistence of release of the foreign material can be used as various products, such as foods, pharmaceuticals, quasi-pharmaceuticals, cosmetics and miscellaneous goods depending on the foreign material enclosed therein. For example, an edible flavoring-encapsulated microcapsule of the present invention can be used as a flavoring ingredient in chewing gum. A chewing gum that contains the microcapsule as a flavoring ingredient exhibits the effects of, depending on the type of saccharide and polyhdric alcohols adhered, promptly releasing the flavor in the mouth, releasing the flavor intensely in the mouth, and maintaining the flavor/taste for a long time of period.

1. Microcapsules having at least one foreign substance encapsulated in microorganism cells onto whose surfaces are adhered at least one member selected from the group consisting of saccharides, high-intensity sweeteners, proteins and polyhydric alcohols.

2. The microcapsules according to claim 1, wherein the microorganism cells are that have been previously treated to remove the endogenous intracellular components.

3. The microcapsules according to claim 1, wherein the microorganism cells are yeast cells.

4. The microcapsules according to claim 1, wherein the saccharides are at least one member selected from the group consisting of monosaccharides, disaccharides, trisaccharides, tetrasaccharides, oligosaccharides, polysaccharides and sugar alcohols.

5. The microcapsules according to claim 1, wherein to the surfaces of the microorganism cells are adhered at least one member selected from the group consisting of lactose, maltitose, trehalose, palatinit, dextrin, glycerin, propylene glycol, polyglycerin, and polypropylene glycol.

6. The microcapsules according to claim 1, wherein the foreign substance encapsulated in the microorganism cells is hydrophobic substance.

7. The microcapsules according to claim 1, wherein the foreign substance encapsulated in the microorganism cells is at least one member selected from the group consisting of flavorings, colorings, acidulants, seasonings, bitterants, spices, sweeteners, fats/lipids, fatty acids, amino acids, enzymes, vitamins, animal and plant extracts, and bioactive substances.

8. The microcapsules according to claim 1, which is used as a constituent in foods, pharmaceuticals, quasi-pharmaceuticals, cosmetics or feeds.

9. A manufacturing method of microcapsule comprising the steps of:
(1) encapsulating a foreign substance inside microorganism cells, and
(2) adhering onto the surface of the microorganism cells obtained in step (1) at least one member selected from the group consisting of saccharides, high-intensity sweeteners, proteins and polyhydric alcohols.

10. The manufacturing method of microcapsule according to claim 9, wherein step (1) is a step of encapsulating a foreign substance inside microorganism cells whose endogenous intracellular components have been removed.

11. An oral composition containing microcapsules of claim 1.

12. The oral composition according to claim 11, which contains the microcapsules of claim 1 as flavorings, colorings, seasonings, acidulants, bitterants, spices or sweeteners.

13. The oral composition according to claim 11, which is held or chewed in the mouth for a prolonged time.

14. The oral composition according to claim 11, which is a chewing gum.

15. A method for controlling the speed, intensity or persistence of the foreign substances released from microcapsules of foreign substances encapsulated in microorganism cells, comprising the step of adhering onto the surface of these microcapsules at least one member selected from the group consisting of saccharides, high-intensity sweeteners, proteins and polyhydric alcohols.

16. The method according to claim 15, which is a method for controlling the speed, intensity or persistence of the expression of the flavor/taste from microcapsules of a flavor/taste component encapsulated in the microorganism cells.

17. The method according to claim 16, wherein the microcapsules of the flavor/taste component encapsulated in the microorganism cells are obtained by encapsulating a flavor/taste component in microorganism cells whose endogenous intracellular components have been removed.

18. The method according to claim 15, which is a method for increasing the speed, intensity or persistence of the expression of the flavor/taste from the microcapsules.

19. A method for controlling the speed, intensity and persistence of the expression of flavor/taste in an oral composition including microcapsules of flavor/taste components encapsulated inside microorganism cells onto whose surfaces are adhered at least one member selected from the group consisting of saccharides, high-intensity sweeteners, proteins and polyhydric alcohols.

20. The method according to claim 19, wherein the microorganism cells are that have been removed the endogenous intracellular components.

21. The method according to claim 19, which is a method for increasing the speed, intensity and persistence of the expression of flavor/taste in an oral composition.

22. Use of microcapsules having foreign substances encapsulated inside microorganism cells onto whose surfaces are adhered at least one member selected from the group consisting of saccharides, high-intensity sweeteners, proteins and polyhydric alcohols, as flavorings, sweeteners, colorings, seasonings, bitterants, spices or acidulants.

23. Use of microcapsules having foreign substances encapsulated inside microorganism cells onto whose surfaces are adhered at least one member selected from the group consisting of saccharides, high-intensity sweeteners, proteins and polyhydric alcohols, for the preparation of an oral composition.

24. Use of microcapsules having foreign substances encapsulated inside microorganism cells onto whose surfaces are adhered at least one member selected from the group consisting of saccharides, high-intensity sweeteners, proteins and polyhydric alcohols, for the preparation of an oral composition having an improved speed, intensity and persistence of the expression of flavor/taste inside the mouth.

25. Use of microcapsules having foreign substances encapsulated inside microorganism cells onto whose surfaces are adhered at least one member selected from the group consisting of saccharides, high-intensity sweeteners, proteins and polyhydric alcohols, for increasing the speed, intensity and persistence of the expression of flavor/taste in an oral composition.
